(19) United States
(12) Patent Application Publication
(10) Pub. No.: US 2008/0064899 A1
(43) Pub. Date: Mar. 13, 2008

(54) Method For Producing Alpha, Beta-Unsaturated Carboxylic Acid

(76) Inventors: Seiichi Kawato, Hiroshima (JP); Akio Takeda, Hiroshima (JP); Yoshiyuki Himeno, Hiroshima (JP)

Correspondence Address: OBLON, SPIVAK, MCCLELLAND MAIER & NEUSTADT, P.C., 1940 DUKE STREET, ALEXANDRIA, VA, 22314, US

(21) Appl. No.: 11/628,215

(22) PCT Filed: May 30, 2005
(86) PCT No.: PCT/JP05/09857

§ 371 Date: Dec. 1, 2006

(30) Foreign Application Priority Data
Jun. 2, 2004 (JP) 2004-164561

(51) Int. Cl. C07C 51/25

(52) U.S. Cl. 562/533

(57) ABSTRACT

The present invention provides a method for producing an α,β-unsaturated carboxylic acid in high yield. In particular, the present invention resides in a method for producing an α,β-unsaturated carboxylic acid by oxidation of an olefin or an α,β-unsaturated aldehyde with molecular oxygen in liquid phase in the presence of a catalyst containing at least palladium, wherein at least one compound selected from the group consisting of p-methoxyphenol, 4,4′-dihydroxytetraphenylmethane, 1,1,1-tris(p-hydroxyphenyl)ethane, compounds having an N-oxyl group in the molecule and compounds having an N-nitrosyl group in the molecule is caused to be coexistent.

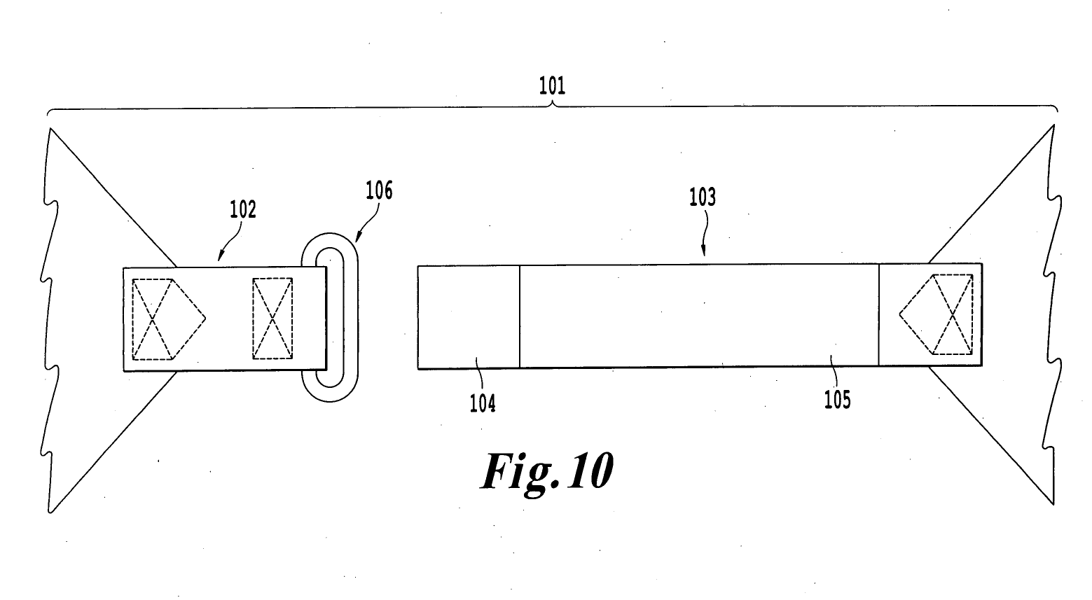

### TECHNICAL FIELD

The present invention relates to a method for producing an α,β-unsaturated carboxylic acid by oxidation of an olefin or an α,β-unsaturated aldehyde with molecular oxygen in liquid phase in the presence of a catalyst containing at least palladium.

### BACKGROUND ART

Methods for producing an α,β-unsaturated carboxylic acid by oxidation of an olefin or an α,β-unsaturated aldehyde with molecular oxygen in liquid phase in the presence of a catalyst containing at least palladium have been proposed in various documents such as Patent Documents 1 to 3. These methods are characterized in that an olefin or an α,β-unsaturated aldehyde is oxidized with molecular oxygen in liquid phase, using a palladium catalyst prepared in various methods.
Patent Document 1: International Publication Pamphlet No. WO 02/083,299 A1.
Patent Document 2: Japanese Patent Application Laid-Open No. 60-155,148.
Patent Document 3: Japanese Patent Application Laid-Open No. 60-139,341.

### DISCLOSURE OF INVENTION

### Problem to be Solved by the Invention

However, when the present inventors produced an α,β-unsaturated carboxylic acid from an olefin or an α,β-unsaturated aldehyde according to the methods described in Examples of Patent Documents 1 to 3, it was found that by-products such as various polymers and oligomers were produced in large amount in addition to the by-products described in Patent Documents 1 to 3. In Patent Documents 1 to 3, these polymers and oligomers were not captured, and hence the actual selectivities to the α,β-unsaturated carboxylic acid in view of these by-products were found to be lower than those described in Examples of Patent Documents 1 to 3.

In Patent Document 2, a method was further disclosed in which the reaction was carried out in the presence of free radical inhibitors such as butylated hydoxytoluene and 2,2′-methylene-bis(4-methyl-6-tert-butylphenol) in the reaction system, and in this case, it was described that the selectivity to the α,β-unsaturated carboxylic acid was improved. However, it was found that the reaction from an olefin or an α,β-unsaturated aldehyde to an α,β-unsaturated carboxylic acid was considerably inhibited and the reaction activity is lowered, in some reaction conditions, and hence the yield of the α,β-unsaturated carboxylic acid was lowered.

Consequently, the methods for producing α,β-unsaturated carboxylic acids described in Patent Documents 1 to 3 were not sufficient yet and a method with higher yield has been desired.

It is an object of the present invention to provide a method for producing an α,β-unsaturated carboxylic acid with high yield.

### Means for Solving Problem

The present invention resides in a method for producing an α,β-unsaturated carboxylic acid by oxidation of an olefin or an α,β-unsaturated aldehyde with molecular oxygen in liquid phase in the presence of a catalyst containing at least palladium, wherein at least one compound selected from the group consisting of p-methoxyphenol, 4,4′-dihydroxytetraphenylmethane, 1,1,1-tris(p-hydroxyphenyl)ethane, compounds having an N-oxyl group in the molecule and compounds having an N-nitrosyl group in the molecule is caused to be coexistent.

### Effect of the Invention

According to the present invention, an α,β-unsaturated carboxylic acid can be produced in high yield when produced by oxidation of an olefin or an α,β-unsaturated aldehyde with molecular oxygen in liquid phase in the presence of a catalyst containing at least palladium.

### BEST MODE FOR CARRYING OUT THE INVENTION

The method for producing an α,β-unsaturated carboxylic acid of the present invention is a method for producing an α,β-unsaturated carboxylic acid by oxidation of an olefin or an α,β-unsaturated aldehyde with molecular oxygen in liquid phase in the presence of a catalyst containing at least palladium, wherein at least one compound selected from the group consisting of p-methoxyphenol, 4,4′-dihydroxytetraphenylmethane, 1,1,1-tris(p-hydroxyphenyl)ethane, compounds having an N-oxyl group in the molecule and compounds having an N-nitrosyl group in the molecule is caused to be coexistent.

Hereinafter, the method for producing an α,β-unsaturated carboxylic acid of the present invention will be explained in detail.

The catalyst to be used in the present invention necessarily contains at least palladium, however, it may contain an element other than palladium. The kind of the element other than palladium is not particularly limited, and it may include platinum, rhodium, ruthenium, iridium, gold, lead, thallium, mercury, carbon, or the like. Palladium is contained in the catalyst (in the case of a supported type catalyst which will be mentioned later, this means the portion except the carrier) preferably with the amount of 25% by mass or more.

The catalyst can be produced, for example, by contacting a palladium compound with a reducing agent to reduce the palladium compound. In the case of producing a catalyst that contains an element other than palladium, the method for producing the catalyst is not particularly limited, and for example, a method of reducing the compound which contains the element other than palladium by causing it to coexist with the palladium compound, or a method of reducing the compound which contains the element other than palladium by causing it to coexist with metal palladium prepared by previously contacting the palladium compound with a reducing agent can be used.

The palladium compound is not particularly limited, and palladium chloride, palladium oxide, palladium acetate, palladium nitrate, palladium sulfate, palladium ammonium chloride, palladium tetraammine complex, palladium acetylacetonate complex, palladium alloy, or the like can be used. A catalyst precursor on which the palladium compound is supported or impregnated can also be used.

The reducing agent is not particularly limited, and hydrogen, hydrazine, formaldehyde, ethanol, sodium borohydride, a compound having double bond, or the like can be used. The compounds having double bond includes propylene, isobutylene, allyl alcohol, methallyl alcohol, acrolein, methacrolein, acrylic acid, methacrylic acid. As the method for contacting the palladium compound with the reducing agent, condition of gas phase or liquid phase may be adopted depending on the state of the reducing agent, or both of the conditions of contacting in liquid phase and in gas phase may jointly be adopted.

The catalyst is preferably a supported catalyst in which catalyst-composing elements including at least palladium are supported on a carrier in order to increase the surface area of the catalyst-composing elements to realize the high dispersion of the catalyst-composing elements, and/or in order to control the acidic or basic sites of the catalyst. However, the catalyst is not necessarily the supported one, and it may be the one that is merely composed of catalyst-composing elements including at least palladium. As the carrier, activated carbon, carbon black, silica, alumina, magnesia, calcia, titania, zirconia, or the like can be used.

When the carrier is used, the palladium loading ratio of the supported catalyst is preferably 0.1% by mass or more to the carrier before it is supported, more preferably 0.5% by mass or more, furthermore preferably 1% by mass or more. The palladium loading ratio is preferably 30% by mass or less to the carrier before it is supported, more preferably 20% by mass or less, furthermore preferably 15% by mass or less.

The catalyst may be activated before it is served to the reaction. The method of activation is not particularly limited, and various methods can be used. Heating in the hydrogen flow under reducing atmosphere is ordinarily used as the method of activation.

In the presence of the catalyst containing at least palladium as mentioned above, an α,β-unsaturated carboxylic acid is produced by oxidation of an olefin or an α,β-unsaturated aldehyde with molecular oxygen in liquid phase.

The production of an α,β-unsaturated carboxylic acid may be carried out by any one of a continuous type operation and a batch type operation, however, a continuous type operation is industrially preferable in view of the productivity.

The raw olefins include propylene, isobutylene, and 2-butene. Among them, propylene and isobutylene are suitable. The raw α,β-unsaturated aldehydes include acrolein, methacrolein, crotonaldehyde (β-methylacrolein), cinnamaldehyde (β-phenylacrolein). Among them, acrolein and methacrolein are suitable. The raw olefin or the raw α,β-unsaturated aldehyde may contain a small amount of a saturated hydrocarbon and/or a lower saturated aldehyde as impurities.

The α,β-unsaturated carboxylic acid to be produced is, in the case that the raw material is an olefin, the one having the same carbon skeleton as the olefin has, and, in the case that the raw material is an α,β-unsaturated aldehyde, the one in which the aldehyde group in the α,β-unsaturated aldehyde has changed into the carboxyl group.

As the reaction solvent to be used in the production of the α,β-unsaturated carboxylic acid, for example, it is preferable to use at least one compound selected from the group consisting of tertiary butanol, cyclohexanol, acetone, methyl ethyl ketone, methyl isobutyl ketone, acetic acid, propionic acid, n-butyric acid, iso-butyric acid, n-valeric acid, iso-valeric acid, ethyl acetate, methyl propionate, hexane, cyclohexane and toluene. Among them, at least one compound selected from the group consisting of tertiary butanol, methyl isobutyl ketone, acetic acid, propionic acid, n-butyric acid, iso-butyric acid, n-valeric acid and iso-valeric acid is more preferable. Further, causing water to coexist with the organic solvent is preferable to produce the α,β-unsaturated carboxylic acid in high selectivity. The amount of water to be coexistent is not particularly limited, however, it is preferably 2% by mass or more to the total mass of the organic solvent and water, more preferably 5% by mass or more. This amount is preferably 70% by mass or less, more preferably 50% by mass or less. The mixture of the organic solvent and water is preferably homogeneous, however it may be heterogeneous.

In the method for producing an α,β-unsaturated carboxylic acid of the present invention, it is important that at least one compound selected from the group consisting of p-methoxyphenol, 4,4′-dihydroxytetraphenylmethane, 1,1,1-tris(p-hydroxyphenyl)ethane, compounds having an N-oxyl group in the molecule and compounds having an N-nitrosyl group in the molecule is caused to be coexistent. The yield of producing an α,β-unsaturated carboxylic acid from an olefin or an α,β-unsaturated aldehyde can be increased by causing the compound to coexist. The compound is supposed not only to have the function that it effectively suppress the polymerization of the α,β-unsaturated carboxylic acid produced but also to maintain or improve the activity of the reaction from the olefin or the α,β-unsaturated aldehyde to the α,β-unsaturated carboxylic acid.

The compound to be coexistent with the catalyst is preferably at least one compound selected from the group consisting of p-methoxyphenol, 4,4′-dihydroxytetraphenylmethane and compounds having an N-oxyl group in the molecule, more preferably either one or both of p-methoxyphenol and 4,4′-dihydroxytetraphenylmethane.

The compounds having an N-oxyl group in the molecule include
2,2,6,6-tetramethylpiperidine-N-oxyl,
4-hydroxy-2,2,6,6-tetramethylpiperidine-N-oxyl,
4-acetylamino-2,2,6,6-tetramethylpiperidine-N-oxyl,
4-benzoylamino-2,2,6,6-tetramethylpiperidine-N-oxyl,
4-propionylamino-2,2,6,6-tetramethylpiperidine-N-oxyl,
4-butyrylamino-2,2,6,6-tetramethylpiperidine-N-oxyl,
4-acetyloxy-2,2,6,6-tetramethylpiperidine-N-oxyl,
4-benzoyloxy-2,2,6,6-tetramethylpiperidine-N-oxyl,
4-methoxy-2,2,6,6-tetramethylpiperidine-N-oxyl,
4-ethoxy-2,2,6,6-tetramethylpiperidine-N-oxyl,
2,2,6,6-tetramethyl-4-piperidone-N-oxyl,
3-carbamoyl-2,2,5,5-tetramethyl-pyrrolidine-N-oxyl. Among them,
4-hydroxy-2,2,6,6-tetramethylpiperidine-N-oxyl,
4-acetylamino-2,2,6,6-tetramethylpiperidine-N-oxyl or
4-acetyloxy-2,2,6,6-tetramethylpiperidine-N-oxyl is preferable.

Further, the compounds having an N-nitrosyl group in the molecule include N-nitrosophenylamine, N-nitrosodiphenylamine, N-nitroso-cyclohexylhydroxylamine, N-nitrosophenylhydroxylamine and salts of these compounds. Among them, N-nitrosodiphenylamine or N-nitrosophenylhydroxylamine ammonium salt is preferable.

The detailed mechanism about why the α,β-unsaturated carboxylic acid can be produced in high yield by causing these compounds to coexist is unclear, however, it is presumed that these compounds increase the selectivity to the α,β-unsaturated carboxylic acid by suppressing the production of by-products, playing the role as free radical inhibitors which capture free radicals that cause the polymerized by-products, as well as that the interaction of these compounds and palladium atom which is the main component of the catalyst causes the reaction activity to increase to obtain the α,β-unsaturated carboxylic acid in high yield.

The amount of use of these compounds is preferably 0.001 part by mass or more to 100 parts by mass of the raw olefin or the raw α,β-unsaturated aldehyde, more preferably 0.005 part by mass or more, because the effect of increasing the yield is more prominent as the amount of use increases in the region where the amount of use is small. Further, in the region where the amount of use is large, it is hard to realize the effect of further increasing the yield by increasing the amount of use, and hence from the economical point of view, the amount of use is preferably 5 parts by mass or less, more preferably 1 part by mass or less. These compounds can be used alone or in combination of plural kinds depending on the composition of the reaction solvent. Further, it is possible to use other compounds, as long as these compounds do not suppress the reaction, together with the above-mentioned compounds, because, by using the other compounds additionally, there is a case of an occurrence of the improved effect that the production of polymerized by-products is more suppressed in comparison with the case of using only the above-mentioned compounds.

The other compounds which can be used together with the above-mentioned compounds include hydroquinone; butylated hydroxytoluenes such as 2,6-di-t-butyl-p-cresol; sulfur-containing compounds such as phenothiazine; and amine-containing compounds such as N-phenyl-N′-isopropyl-p-phenylenediamine.

The concentration of the olefin or the α,β-unsaturated aldehyde which is the raw material of the reaction is preferably 0.1% by mass or more to the solvent existing in the reactor, more preferably 0.5% by mass or more. This concentration is preferably 30% by mass or less, more preferably 20% by mass or less.

As the source of molecular oxygen to be used in the production of the α,β-unsaturated carboxylic acid, air is economical, however, pure oxygen or a mixed gas of pure oxygen and air can be used, and if necessary, a mixed gas in which air or pure oxygen is diluted with nitrogen, carbon dioxide, water vapor, or the like can also be used.

The amount of molecular oxygen is preferably 0.1 mole or more to 1 mole of the raw olefin or the raw α,β-unsaturated aldehyde, more preferably 0.3 mole or more and furthermore preferably 0.5 mole or more. The amount is preferably 20 mole or less, more preferably 15 mole or less and furthermore preferably 10 mole or less.

Ordinarily, the catalyst is used in a suspended state in the reaction liquid, however, it may be used in a fixed bed. The amount of use of the catalyst is preferably 0.1% by mass or more to the solution existing in the reactor, more preferably 0.5% by mass or more and furthermore preferably 1% by mass or more. This amount of use is preferably 30% by mass or less to the solution existing in the reactor, more preferably 20% by mass or less and furthermore preferably 15% by mass or less.

The reaction temperature and the reaction pressure are properly selected depending on the solvent and the raw materials for the reaction to be used. The reaction temperature is preferably 30° C. or more, more preferably 50° C. or more. This temperature is preferably 200° C. or less, more preferably 150° C. or less. Further, the reaction pressure is preferably 0 MPa or more (gauge pressure; hereinafter, every pressure is expressed in gauge pressure), more preferably 0.5 MPa or more. This pressure is preferably 10 MPa or less, more preferably 5 MPa or less.

### EXAMPLES

Hereinafter, the present invention will be more concretely explained with reference to Examples and Comparative Examples, however, the present invention is not limited to these Examples.

The term “part(s)” in the following Examples and Comparative Examples means part(s) by mass, and analysis of the raw materials and the products were carried out using gas chromatography.

When isobutylene was used as the raw material, conversion of isobutylene, selectivity to methacrolein to be produced, selectivity to and yield of methacrylic acid to be produced are defined as follows:

| | |
|---|---|
| Conversion of isobutylene (%) = | (B/A) × 100; |
| Selectivity to methacrolein (%) = | (C/B) × 100; |
| Selectivity to methacrylic acid (%) = | (D/B) × 100; and |
| Yield of methacrylic acid (%) = | (D/A) × 100. |

In these formulae, A represents mole number of isobutylene supplied, B represents mole number of isobutylene reacted, C represents mole number of methacrolein produced and D represents mole number of methacrylic acid produced.

Further, when methacrolein was used as the raw material, conversion of methacrolein, selectivity to and yield of methacrylic acid to be produced are defined as follows:

| | |
|---|---|
| Conversion of methacrolein (%) = | (F/E) × 100; |
| Selectivity to methacrylic acid (%) = | (G/F) × 100; and |
| Yield of methacrylic acid (%) = | (G/E) × 100. |

In these formulae, E represents mole number of methacrolein supplied, F represents mole number of methacrolein reacted and G represents mole number of methacrylic acid produced.

### Example 1

(Catalyst Preparation)

To an autoclave equipped with a stirring blade, 51 parts of acetic acid, 9 parts of water and 1.1 parts of palladium acetate were fed and dissolved under heating at 80° C. while stirred, and the resultant system was cooled to 10° C., and 5.0 parts of an activated carbon (specific surface area; 840 m²/g) as a carrier was added to it and the autoclave was shut tight. Stirring was started at 500 revolutions per minute and gas-phase portion of the autoclave was replaced by nitrogen and 0.6 MPa of propylene gas was introduced. Subsequently, the resultant system was heated to 70° C., stirred at 70° C. for 1 hour and stirring was stopped and the system was cooled to room temperature and the autoclave was opened and the reaction liquid containing precipitate was taken out and the precipitate was filtrated from the reaction liquid under nitrogen flow and washed with hot water. The precipitate thus obtained was dried overnight at 100° C. under nitrogen flow to obtain an activated carbon-supported palladium catalyst. Palladium loading ratio of the catalyst was 10% by mass.

(Evaluation of Reaction)

To an autoclave equipped with a stirring blade, 75 parts of acetone and 25 parts of water were fed as a reaction solvent and 5.5 parts of the activated carbon-supported palladium catalyst obtained by above-mentioned method and 0.02 part of p-methoxyphenol were added and the autoclave was shut tight. Subsequently, gas-phase portion of the autoclave was replaced by nitrogen and 6.5 parts of liquefied isobutylene was introduced into it and the system was stirred at 1,000 revolutions per minute and heated to 90° C. When the heating was finished, air was introduced into the autoclave to the internal pressure of 3.2 MPa. Oxidation reaction of isobutylene was carried out for 60 minutes under this state.

After the reaction was finished, the inside of the autoclave was cooled to 10° C. by ice bath. A gas-sampling bag was attached to the gas outlet of the autoclave and the gas outlet was opened and the emerging gas was collected while the internal pressure of the reactor was released. The reaction liquid containing catalyst was taken out from the autoclave and the catalyst was separated with membrane filter (pore diameter; 0.5 μm) and only the reaction liquid was recovered. The recovered reaction liquid and the sampled gas were analyzed by gas chromatography.

As the result, the conversion of isobutylene of 77.3%, the selectivity to methacrolein of 42.0%, the selectivity to methacrylic acid of 26.1% and the yield of methacrylic acid of 20.2% were obtained.

### Example 2

The same procedure of the catalyst preparation and the evaluation of reaction as in Example 1 was carried out except that p-methoxyphenol was changed to 4-acetylamino-2,2,6,6-tetramethylpiperidine-N-oxyl. As the result, the conversion of isobutylene of 75.8%, the selectivity to methacrolein of 46.0%, the selectivity to methacrylic acid of 20.2% and the yield of methacrylic acid of 15.3% were obtained.

### Example 3

The same procedure of the catalyst preparation and the evaluation of reaction as in Example 1 was carried out except that p-methoxyphenol was changed to 4,4′-dihydroxytetraphenylmethane. As the result, the conversion of isobutylene of 77.1%, the selectivity to methacrolein of 48.8%, the selectivity to methacrylic acid of 22.4% and the yield of methacrylic acid of 17.3% were obtained.

### Comparative Example 1

The same procedure of the catalyst preparation and the evaluation of reaction as in Example 1 was carried out except that p-methoxyphenol was changed to hydroquinone. As the result, the conversion of isobutylene of 42.8%, the selectivity to methacrolein of 66.1%, the selectivity to methacrylic acid of 16.3% and the yield of methacrylic acid of 7.0% were obtained.

### Comparative Example 2

The same procedure of the catalyst preparation and the evaluation of reaction as in Example 1 was carried out except that p-methoxyphenol was not added. As the result, the conversion of isobutylene of 77.8%, the selectivity to methacrolein of 23.6%, the selectivity to methacrylic acid of 9.1% and the yield of methacrylic acid of 7.1% were obtained.

TABLE 1

| | | Conversion of | Selectivity to | Selectivity to | Yield of |
|---|---|---|---|---|---|
| | | isobutylene | methacrolein | methacrylic | methacrylic |
| | Additives | (%) | (%) | acid (%) | acid (%) |
| Ex. 1 | p-methoxyphenol | 77.3 | 42.0 | 26.1 | 20.2 |
| Ex. 2 | 4-acetylamino-2,2,6,6- | 75.8 | 46.0 | 20.2 | 15.3 |
| | tetramethylpiperidine-N-oxyl | | | | |
| Ex. 3 | 4,4′-dihydroxytetraphenylmethane | 77.1 | 48.8 | 22.4 | 17.3 |
| Comp. Ex. 1 | hydroquinone | 42.8 | 66.1 | 16.3 | 7.0 |
| Comp. Ex. 2 | none | 77.8 | 23.6 | 9.1 | 7.1 |

### Example 4

To an autoclave equipped with a stirring blade, 75 parts of acetic acid and 25 parts of water were fed as a reaction solvent and 5.5 parts of the activated carbon-supported palladium catalyst prepared by the same procedure as in Example 1, 2.5 parts of methacrolein and 0.02 part of p-methoxyphenol were added and the autoclave was shut tight. Subsequently, gas-phase portion of the autoclave was replaced by nitrogen and the system was stirred at 1,000 revolutions per minute and heated to 90° C. When the heating was finished, air was introduced into the autoclave to the internal pressure of 3.2 MPa. Oxidation reaction of methacrolein was carried out for 20 minutes under this state.

After the reaction was finished, the inside of the autoclave was cooled to 20° C. by ice bath. A gas-sampling bag was attached to the gas outlet of the autoclave and the gas outlet was opened and the emerging gas was collected while the internal pressure of the reactor was released. The reaction liquid containing catalyst was taken out from the autoclave and the catalyst was separated by centrifugation and only the reaction liquid was recovered. The recovered reaction liquid and the sampled gas were analyzed by gas chromatography.

As the result, the conversion of methacrolein of 84.9%, the selectivity to methacrylic acid of 73.9% and the yield of methacrylic acid of 62.7% were obtained.

### Example 5

The same procedure of the catalyst preparation and the evaluation of reaction as in Example 4 was carried out except that p-methoxyphenol was changed to 4-acetylamino-2,2,6,6-tetramethylpiperidine-N-oxyl. As the result, the conversion of methacrolein of 80.0%, the selectivity to methacrylic acid of 74.2% and the yield of methacrylic acid of 59.4% were obtained.

### Example 6

The same procedure of the catalyst preparation and the evaluation of reaction as in Example 4 was carried out except that p-methoxyphenol was changed to 4-acetyloxy-2,2,6,6-tetramethylpiperidine-N-oxyl. As the result, the conversion of methacrolein of 88.8%, the selectivity to methacrylic acid of 72.2% and the yield of methacrylic acid of 64.1% were obtained.

### Example 7

The same procedure of the catalyst preparation and the evaluation of reaction as in Example 4 was carried out except that p-methoxyphenol was changed to 4,4′-dihydroxytetraphenylmethane. As the result, the conversion of methacrolein of 84.9%, the selectivity to methacrylic acid of 71.6% and the yield of methacrylic acid of 60.8% were obtained.

### Example 8

The same procedure of the catalyst preparation and the evaluation of reaction as in Example 4 was carried out except that p-methoxyphenol was changed to 1,1,1-tris(parahydroxyphenyl)ethane. As the result, the conversion of methacrolein of 79.2%, the selectivity to methacrylic acid of 68.7% and the yield of methacrylic acid of 54.4% were obtained.

### Example 9

The same procedure of the catalyst preparation and the evaluation of reaction as in Example 4 was carried out except that p-methoxyphenol was changed to N-nitrosophenylhydroxylamine ammonium salt. As the result, the conversion of methacrolein of 75.9%, the selectivity to methacrylic acid of 73.1% and the yield of methacrylic acid of 55.5% were obtained.

### Comparative Example 3

The same procedure of the catalyst preparation and the evaluation of reaction as in Example 4 was carried out except that p-methoxyphenol was changed to hydroquinone. As the result, the conversion of methacrolein of 64.5%, the selectivity to methacrylic acid of 70.3% and the yield of methacrylic acid of 45.3% were obtained.

### Comparative Example 4

The same procedure of the catalyst preparation and the evaluation of reaction as in Example 4 was carried out except that p-methoxyphenol was changed to 2,6-di-t-butyl-p-cresol. As the result, the conversion of methacrolein of 69.0%, the selectivity to methacrylic acid of 72.2% and the yield of methacrylic acid of 49.8% were obtained.

### Comparative Example 5

The same procedure of the catalyst preparation and the evaluation of reaction as in Example 4 was carried out except that p-methoxyphenol was changed to phenothiazine. As the result, the conversion of methacrolein of 38.6%, the selectivity to methacrylic acid of 77.3% and the yield of methacrylic acid of 29.8% were obtained.

### Comparative Example 6

The same procedure of the catalyst preparation and the evaluation of reaction as in Example 4 was carried out except that p-methoxyphenol was changed to N-phenyl-N′-isopropyl-p-phenylenediamine. As the result, the conversion of methacrolein of 23.6%, the selectivity to methacrylic acid of 15.5% and the yield of methacrylic acid of 3.7% were obtained.

### Comparative Example 7

The same procedure of the catalyst preparation and the evaluation of reaction as In Example 4 was carried out except that p-methoxyphenol was not added. As the result, the conversion of methacrolein of 90.3%, the selectivity to methacrylic acid of 28.3% and the yield of methacrylic acid of 25.6% were obtained.

TABLE 2

| | | Conversion of | Selectivity to | Yield of |
|---|---|---|---|---|
| | | methacrolein | methacrylic | methacrylic |
| | Additives | (%) | acid (%) | acid (%) |
| Ex. 4 | p-methoxyphenol | 84.9 | 73.9 | 62.7 |
| Ex. 5 | 4-acetylamino-2,2,6,6- | 80.0 | 74.2 | 59.4 |
| | tetramethylpiperidine-N-oxyl | | | |
| Ex. 6 | 4-acetyloxy-2,2,6,6- | 88.8 | 72.2 | 64.1 |
| | tetramethylpiperidine-N-oxyl | | | |
| Ex. 7 | 4,4′-dihydroxytetraphenylmethane | 84.9 | 71.6 | 60.8 |
| Ex. 8 | 1,1,1-tris(parahydroxyphenyl)ethane | 79.2 | 68.7 | 54.4 |
| Ex. 9 | N-nitrosophenylhydroxylamine | 75.9 | 73.1 | 55.5 |
| | ammonium salt | | | |
| Comp. Ex. 3 | hydroquinone | 64.5 | 70.3 | 45.3 |
| Comp. Ex. 4 | 2,6-di-t-butyl-p-cresol | 69.0 | 72.2 | 49.8 |
| Comp. Ex. 5 | phenothiazine | 38.6 | 77.3 | 29.8 |
| Comp. Ex. 6 | N-phenyl-N′-isopropyl- | 23.6 | 15.5 | 3.7 |
| | p-phenylenediamine | | | |
| Comp Ex. 7 | none | 90.3 | 28.3 | 25.6 |

As mentioned above, it was found that an α,β-unsaturated carboxylic acid can be produced in high yield according to the present invention.

What is claimed is:

1. A method for producing an α,β-unsaturated carboxylic acid by oxidation of an olefin or an α,β-unsaturated aldehyde with molecular oxygen in liquid phase In the presence of a catalyst containing at least palladium, wherein at least one compound selected from the group consisting of p-methoxyphenol, 4,4′-dihydroxytetraphenylmethane, 1,1,1-tris(p-hydroxyphenyl)ethane, compounds having an N-oxyl group in the molecule and compounds having an N-nitrosyl group in the molecule is caused to be coexistent.
